(19) United States
(12) Patent Application Publication
(10) Pub. No.: US 2024/0139151 A1
(43) Pub. Date: May 2, 2024

(54) COMPOSITION INCLUDING DECURSINOL AS ACTIVE INGREDIENT FOR PREVENTING OR TREATING SMOOTH MUSCLE CELL PROLIFERATIVE DISEASES

(71) Applicant: KYUNGSUNG UNIVERSITY INDUSTRY COOPERATION FOUNDATION, Busan (KR)

(72) Inventors: Jae Seon KANG, Busan (KR); Hyung Hoi KIM, Busan (KR); Jae Sung PYO, Busan (KR); Seong Jae LEE, Busan (KR); Ye Jin HWANG, Busan (KR); Hyeong Soo KIM, Busan (KR); Jae Ki CHOI, Busan (KR)

(73) Assignee: KYUNGSUNG UNIVERSITY INDUSTRY COOPERATION FOUNDATION, Busan (KR)

(21) Appl. No.: 18/272,219

(22) PCT Filed: Jan. 11, 2022
(86) PCT No.: PCT/KR2022/000507

§ 371 Date: Jul. 13, 2023

(30) Foreign Application Priority Data
Jan. 13, 2021 (KR) 10-2021-0004607

(51) Int. Cl. A61K 31/37; A61K 9/00; A61K 9/08; A61K 9/20; A61K 36/232; A61P 9/12

(52) U.S. Cl. CPC A61K 31/37; A61K 9/0019; A61K 9/0056; A61K 9/0095; A61K 9/08; A61K 9/2018; A61K 9/2059; A61K 36/232; A61P 9/12

(57) ABSTRACT

The present invention relates to a composition including decursinol as an active ingredient for preventing or treating angina, arteriosclerosis, cerebral infarction, and hypertension. Poorly soluble decursinol was found to improve angina, arteriosclerosis, cerebral infarction, prostate hypertrophy, and diabetic hypertension by inhibiting the overgrowth of vascular endothelial cells, and is expected to be usable in the development of a composition for preventing or treating angina, arteriosclerosis, cerebral infarction, prostate hypertrophy, and diabetic hypertension.

### TECHNICAL FIELD

The present disclosure relates to a composition including decursinol as an active ingredient for inhibiting the proliferation of smooth muscle cells.

### BACKGROUND ART

Population aging is one of the most important social issues in the world. As the number of geriatric diseases is rapidly increasing along with the rapid increase in the aging population, the resulting increase in medical expenses is also emerging as an essential social problem that needs to be solved (Maengje Cho, 2009). In particular, hyperproliferation of vascular smooth muscle cells is a key factor in the progression of atherosclerotic lesions. Excessive exposure to sugar induces vascular endothelial cell dysfunction and overproliferation of vascular smooth muscle cells, leading to diabetic hypertension. In addition, excessive proliferation of smooth muscle cells in the coronary arteries causes angina pectoris, and in severe cases, requires stent insertion for the treatment thereof. These diseases are serious diseases that lower the quality of life and increase health risks. In addition, cardiovascular disease is analyzed as a higher cause of death, compared to the past, and was reported to account for close to 50% of the cause of death.

A fundamental treatment for such diseases is to prevent overgrowth of vascular smooth muscle. When imparted to vascular endothelial cells, damage is recognized through signal transduction and then excessive proliferation of smooth muscle occurs. Inhibition or control is thus necessary for the excessive proliferation of smooth muscle cells.

Korean Patent No. 10-2010-0047544 A discloses a pharmaceutical composition including an Angelica gigas extract as an active ingredient for the prevention or treatment of vascular smooth muscle cell hyperproliferative disease. The root is used as a medicine for various diseases such as pain relief, anticancer, alleviation of kidney toxicity, improvement of liver function, treatment of diabetic hypertension, improvement of blood circulation, etc. Components found in Angelica are known to include decursin, decursinol angelate, nodakenin, umbelliferon, β-sitosterol, α-pinene, limonene, and the like.

Decursinol, which is one of the biologically active ingredients of Angelica, has been, along with decursin and decursinol angelate, found to exhibit antipyretic and analgesic effects, prevention of septic shock, gastric mucosal protection, prevention of atopic dermatitis, inhibition of cancer cell actions, and protection against neurotoxicity. Although decursinol is present in a very small amount in the root of Angelica, it can be obtained in large quantities and with high purity by hydrolyzing decursin or decursinol angelate, which are the main components of Angelica gigas, with a base. Leading to the present disclosure, intensive and thorough research conducted by the present inventors resulted in finding in vivo effects of highly pure decursinol.

Korean Patent No. 10-2004-0079265 A, which is a related art, describes a vascular relaxant containing decursin or decursinol angelate for preventing or treating vascular diseases and Korean Patent No. 1837479 discloses a composition containing a mixture of a red ginseng extract, an angelica extract, and a citrus unshiu peel extract for preventing or treating hyperproliferative diseases of vascular smooth muscle cells, but nowhere is the inhibitory effect of decursinol on vascular smooth muscle cell proliferation described in the documents.

Korean Patent No. 2028334 discloses a composition including a mixture of herbal medicine extracts as an active ingredient for preventing, alleviating, or treating angiogenesis-dependent diseases and Korean Patent No. 10-2019-0052909 discloses a composition containing Houttuynia cordata extract and Angelica gigas extract for treating benign prostatic hyperplasia and a producing method therefor. However, the decursinol composition for inhibiting vascular smooth muscle cell proliferation according to the present disclosure differs from the compositions of the documents in terms of configuration and effect.

### DISCLOSURE OF INVENTION

### Technical Problem

An aspect of the present disclosure aims to provide a composition containing decursinol as an active ingredient for inhibiting the growth of vascular smooth muscle. Another aspect of the present disclosure is to provide a composition for treating diseases attributed to excessive growth of vascular smooth muscle, including angina pectoris, arteriosclerosis, cerebral infarction, benign prostatic hyperplasia, and diabetic hypertension.

### Solution to Problem

The present disclosure provides a composition including decursinol as an active ingredient for inhibiting proliferation of smooth muscle cells. The composition for inhibiting proliferation of smooth muscle cells may be used for treatment of angina pectoris, arteriosclerosis, cerebral infarction, benign prostatic hyperplasia, and diabetic hypertension.

The composition for inhibiting the proliferation of smooth muscle cells may be prepared by a preparing method including a first step of mixing decursinol with a detergent to give a first mixture; a second step of adding ethanol to the first mixture and mixing chitosan therewith to give a second mixture; a third step of dissolving the second mixture by adding a basic amino acid and purified water thereto, followed by adjusting the pH of the solution into 8.6 to give a third mixture; and a fourth step of sterilizing the third mixture.

In the first step, the detergent may be at least one selected from the group consisting of sodium dodecyl sulfate, polyoxyethylene sorbitan monooleate (Tween-80), polyoxyethylene sorbitan monostearate (Tween-60), polyoxyethylene sorbitan monopalmitate (Tween-40), polyoxyethylene sorbitan monolaurate (Tween-20), triton X-100, and nonyl phenoxypolyethoxylethanol (NP-40).

In the second step, the water-soluble chitosan may be chitosan with a molecular weight of 20,000 or less.

In the third step, the basic amino acid may be arginine, lysine, histidine, or a mixture thereof.

The inhibition of excessive growth of vascular smooth muscle may be inhibition of the growth attributed to a high blood sugar level.

The composition for inhibiting proliferation of smooth muscle cells according to the present disclosure may be prepared into a health functional food for alleviating symptoms attributed to the excessive growth of vascular endothelial cells, including angina pectoris, arteriosclerosis, cerebral infarction, benign prostatic hyperplasia, and diabetic hypertension.

Below, a detailed description will be given of the present disclosure.

The angelica extract can be obtained by subjecting Angelica gigas to extraction with one or more solvents selected from the group consisting of water, a lower alcohol of C1 to C4, acetone, hexane, dichloromethane, and ethyl acetate, and the lower alcohol of C1 to C4 may include methanol, ethanol, propanol, isopropanol, butanol, and the like, with preference for ethanol.

In addition, the Angelica extract may be a fraction obtained by re-fractionation with water, a lower alcohol of C1 to C4, or a mixture thereof, and the lower alcohol of C1 to C4 may include methanol, ethanol, isopropanol, and butanol.

The decursinol may be isolated by adding an alkaline solution to the Angelica extract, conducting thermal decomposition, and then neutralizing same with an acidic solution.

The alkaline solution may be a solution of KOH, NaOH, or a mixture thereof, with preference for a KOH solution, but is not limited thereto.

The acidic solution may be a solution of HCl, HNO₃, H₂SO₄, or a mixture thereof, with preference for an HCl solution, but is not limited thereto.

On the other hand, the decursinol of the present disclosure may be synthesized using a typical method in the art and can be prepared into a pharmaceutically acceptable salt.

The formulation may include decursinol solubilized through a first step of mixing decursinol with a detergent to give a first mixture; a second step of adding ethanol to the first mixture and mixing chitosan therewith to give a second mixture; a third step of dissolving the second mixture by adding a basic amino acid and purified water thereto, followed by adjusting the pH of the solution into 8.6 to give a third mixture; and a fourth step of sterilizing the third mixture.

As used herein, the term “solubilization” refers to a process of increasing the solubility of a material that is unlikely to be solved in water. The solubilization method may be used to increase the solubility of decursinol, which is a poorly soluble material.

The detergent in the first step may be at least one selected from the group consisting of sodium dodecyl sulfate, polyoxyethylene sorbitan monooleate, polyoxyethylene sorbitan monostearate, polyoxyethylene sorbitan monopalmitate, polyoxyethylene sorbitan monolaurate, Triton X-100, and nonylphenoxy polyethoxylethanol.

The detergent may be added in an amount of 0.001% by weight relative to the poorly soluble material.

The water-soluble chitosan in the second step is a kind of chitosan with increased solubility and is employed to improve the homogeneity of the composition. The chitosan may be prepared by solubilizing chitosan with a molecular weight of 20,000 or less.

The water-soluble chitosan may be contained in an amount of 0.1-10% [w/v], based on the volume of the pharmaceutical composition. When contained in an amount less than 0.1%, the water-soluble chitosan is difficult to sufficiently disperse without no substantial effects. More than 10% of the water-soluble chitosan brings about overdue viscosity that leads to poor uniformity, and the bulk mass is difficult to mix and economically undesirable.

The basic amino acid in the third step serves as a dissolving agent and may be arginine, lysine, histidine, or a mixture thereof, with preference for arginine or lysine.

The basic amino acid may be contained in an amount of 0.1-4% [w/v] based on the volume of the pharmaceutical composition. At a content less than 0.1% of the basic amino acid, it is difficult to assist the solubility. More than 4% of the basic amino acid brings about overdue viscosity that leads to gelling and the bulk mass is not economically desirable.

The composition for inhibiting the proliferation of smooth muscle cells according to the present invention can be prepared into a herbal pharmaceutical composition for treating symptoms caused by overgrowth of vascular endothelial cells, including angina pectoris, arteriosclerosis, cerebral infarction, prostatic hyperplasia, and diabetic hypertension.

Decursinol may be used preferably in an amount of amount of 0.001 to 50% by weight, more preferably 0.001 to 40% by weight, and most preferably 0.001 to 30% by weight, based on the total weight of the pharmaceutical composition.

The pharmaceutical composition may be formulated as oral dosage forms such as powders, granules, tablets, capsules, suspensions, emulsions, syrups or aerosol formulations, external dosage forms, suppositories, or sterile injectable solutions. Examples of the carrier, excipient or diluent that may be used in the pharmaceutical composition include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, etc. The composition may be formulated with commonly used diluents or excipients, such as fillers, extenders, binders, humectants, disintegrants, surfactants, sweeteners, acidifiers, etc. Solid formulations may be prepared by blending decursinol of the present disclosure with at least one excipient, for example, starch, calcium carbonate, sucrose, lactose, or gelatin. In addition to simple excipients, lubricants such as magnesium stearate or talc may also be used. Suspensions, solutions for internal use, emulsions, syrups, and the like are exemplified as liquid formulations for oral administration. The liquid formulations may contain various excipients, for example, wetting agents, flavoring agents, aromatics, preservatives, and acidifiers in addition to the usually used simple diluents water and liquid paraffin. Formulations for parenteral administration include sterilized aqueous solutions, non-aqueous solutions, suspensions, emulsions, freeze-dried preparations, and suppositories. As non-aqueous solvents or suspending agents, propylene glycol, polyethylene glycol, plant oils such as olive oil, injectable esters such as ethyl oleate, and the like can be used. As the base of the suppositories, witepsol, Macrogol, Tween 61, cacao butter, laurin fat, glycerogelatin and the like can be used.

The dose of the pharmaceutical composition may vary depending on age, gender, and body weight of a subject to be treated, a particular disease or pathological condition to be treated, severity of a disease or pathological condition, route of administration, and determination of a prescriber. The determination of the dose based on these factors is within the level of a person skilled in the art, and the general dose is in the range of approximately 0.01-2,000 mg/kg/day. A more preferable dose is 0.1-500 mg/kg/day. The administration may be carried out once a day or several times in a divided dose a day. The above dose is not intended to restrict the scope of the present disclosure in any way.

The pharmaceutical composition may be administered to mammals, such as rats, domestic animals, and humans, via various routes. All modes of administration may be contemplated, and for example, the administration may be carried out through oral, rectal, intravenous, intramuscular, subcutaneous, endometrial, intracerebroventricular injection or dermal application. Since the compound of the present disclosure has almost no toxicity and side effects, it is a medicinal material that can be safely used even when taken for a long period of time for prophylactic purposes.

In addition, the composition for inhibiting the proliferation of smooth muscle cells of the present disclosure can be prepared into a health functional food for the alleviation of symptoms caused by overgrowth of vascular endothelial cells, including angina pectoris, arteriosclerosis, cerebral infarction, benign prostatic hyperplasia, and diabetic hypertension.

The health functional food may contain a sitologically acceptable food supplementary additive and can be used for alleviating symptoms caused by overgrowth of vascular endothelial cells, including angina pectoris, arteriosclerosis, cerebral infarction, benign prostatic hyperplasia, and diabetic hypertension, by inhibiting the overgrowth of vascular smooth muscle.

The decursinol may be used preferably in an amount of 0.001 to 50% by weight, more preferably in an amount of 0.001 to 30% by weight, and most preferably in an amount of 0.001 to 10% by weight, based on the total weight of the food composition.

The health functional food may be in the form of tablets, capsules, pills, or liquids. Examples of the food to which the extract of the present disclosure can be added include various foods, including beverages, gums, teas, vitamin complexes, health functional foods, etc.

### Advantageous Effects of Invention

The present disclosure is drawn to a composition including decursinol as an active ingredient for inhibiting the proliferation of smooth muscle cells and is expected to contribute to the prophylaxis or therapy of symptoms attributed to excessive proliferation of smooth muscle cells, including angina, arteriosclerosis, cerebral infarction, benign prostatic hyperplasia, and diabetic hypertension.

### BRIEF DESCRIPTION OF DRAWINGS

FIG. 1 shows results of TLC analysis for the purified decursinol matters of the present disclosure: lane 1 for the decursinol with a purity of 99% or higher, used in the present disclosure, lane 2 for the Angelica gigas Nakai extract, lane 3 for the primary purified material of the Angelica gigas Nakai extract, and lane 4 for the secondary purified material of the Angelica gigas Nakai extract.

FIG. 2 shows NMR spectra of standard reference decursinol (A) and the purified decursinol of the present disclosure (B).

FIG. 3 is a plot of the proliferation of the vascular smooth muscle cell line A10 in 5.5 and 30 mM glucose conditions.

FIG. 4 is a graph showing the effects of decursinol on the proliferation of the vascular smooth muscle cell line A10 in 5.5 and 30 mM glucose conditions.

FIG. 5 shows western blots accounting for the effect of decursinol on the expression of phospho-protein kinase C (p-PKC) in the vascular smooth muscle cell line A10 grown in 5.5 and 30 mM glucose conditions.

FIG. 6 is a graph elucidating whether or not the osmotic pressure condition of high sugar affects the proliferation of the vascular smooth muscle cell line A10.

### BEST MODE FOR CARRYING OUT THE INVENTION

Hereinafter, embodiments of the present disclosure will be described in detail with reference to the accompanying drawings. However, the present disclosure is not limited to the embodiments described herein and may be embodied in other forms. Rather, the content introduced herein becomes thorough and complete, and is provided to sufficiently convey the spirit of the present invention to those skilled in the art.

### Example 1: Preparation of Decursinol

### Example 1-1. Preparation of Angelica Extract

Angelica materials used in the Examples were Angelica gigas Nakai native to South and North Korea. Angelica gigas Nakai was finely sectioned into a size of mesh or less and dried to a water content of 5% or less. To the dried Angelica sections, 95% [v/v] ethanol was added in an amount two to four times the weight of the sections. After leaching for 12 hours, the solid matter was filtered off through a filter. The filtrate was dried by evaporation to give a first Angelica concentrate. Ethanol was added in an amount of 1 liter per kg of the first concentrate to obtain a suspension which was then left at −20° C. for 10 hours, followed by centrifugation. The pellet thus formed was removed and the supernatant was dried by evaporation to give a second Angelica concentrate. The second Angelica concentrate was suspended in 50 liters of 60% [v/v] ethanol. Centrifugation separated the 60% [v/v] ethanol layer which was then dried and concentrated by evaporation. The resulting concentrate was suspended in 90% ethanol and centrifuged. The supernatant thus formed was taken and used as a final Angelica gigas extract.

### Example 1-2. Preparation of Decursinol

In 95%[v/v] ethanol was suspended 1,000 g of the Angelica gigas Nakai extract prepared in Example 1-1. The suspension was treated with 5,000 ml of 0.1N KOH and thermally decomposed before neutralization with 575 ml of 6 N HCl. The KCl precipitate formed during the neutralization was removed by centrifugation and the supernatant was left at 4° C. or less until only a small amount of ethanol remained. In the course of concentration, when decursinol, which has poor solubility in ethanol, was precipitated, only the precipitate was separated through filtration and then washed twice with distilled water cooled to about 0-4° C. to afford primary purified decursinol. In addition, this procedure was repetitively carried out to obtain secondary purified decursinol. After at least seven rounds of the procedure, 1,000 g of the decursinol of the present disclosure was acquired.

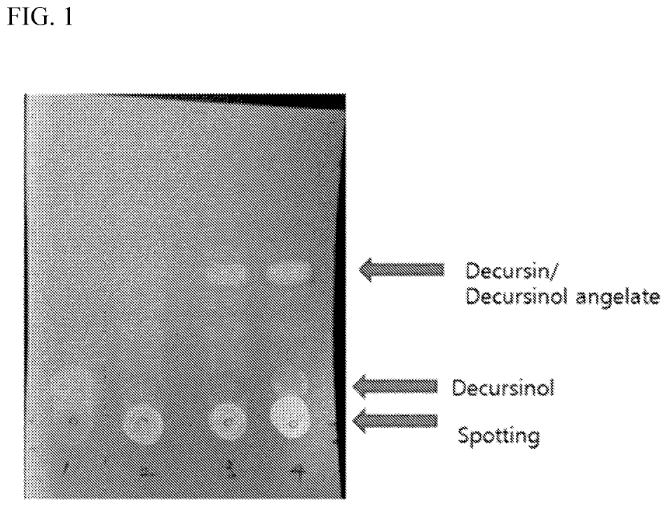

FIG. 1 shows results of thin layer chromatography (TLC) analysis for the purified decursinol matters of the present disclosure: lane 2 was for the Angelica gigas Nakai extract prepared in Example 1-1, lane 3 for the primary purified material of the Angelica gigas Nakai extract prepared in Example 1-1, lane 4 for the secondary purified material of the Angelica gigas Nakai extract, and lane 1 for the decursinol obtained after seven rounds of the thermal decomposition procedure. As can be seen in FIG. 1, the Angelica gigas Nakai extract contained decursin and decursinol angelate at high rates after the purification process. However, almost none of decursin and decursinol angelate were found in the purified material that had undergone repeated rounds of the alkaline and thermal decomposition, indicating that decursin and decursinol angelate were converted into decursinol.

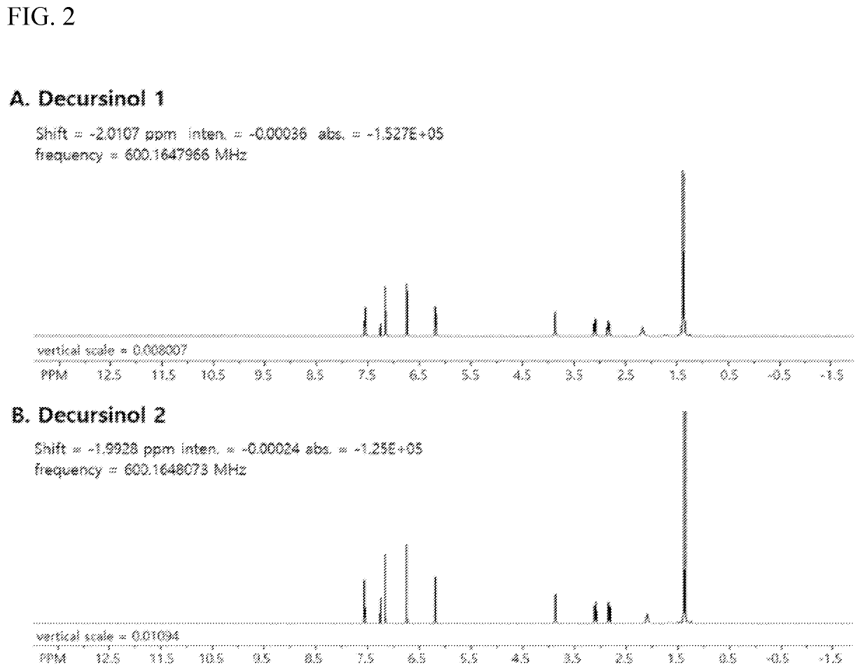

NMR spectra of the high-purified decursinol obtained above and a decursinol standard reference are given in FIG. 2. NMR analysis was performed in the Research Institute for Basic Sciences, Pusan National University. As can be seen, the decursinol obtained through the thermal decomposition procedure of the present disclosure was found to have a purity of 99.9%.

### Example 2: Preparation of Decursinol Solubilized Solution

A solution in which the decursinol of the present disclosure, which is an insoluble component, was solubilized was prepared. In this regard, reference was made to the method disclosed in Korean Patent No. 10-1717672 issued to the present inventors.

Each of 130 μg (ca. 5 μM), 260 μg (ca. 10 μM), and 650 μg (ca. 25 μM) of decursinol was homogeneously mixed with 0.01 g of polyoxyethylene sorbitan monooleate (Tween-80). Then, 1 ml of 95%[v/v] ethanol was added to the mixture and blended with 1.5 g of the basic amino acid lysine or arginine and 90 ml of purified water. The pH was adjusted into 8.6 with 6M HCl or 6M NaOH. Subsequently, purified water was added to form a final volume of 100 ml. Sterilization was made, together with ethanol evaporation, by autoclaving. Thus, a decursinol-solubilized solution was prepared.

### Example 3: Assay for Blood Pressure Safety Test of Angelica Extract and Purified Decursinol

The present inventors have been studying Angelica gigas Nakai for many years while observing the effect of Angelica gigas Nakai extract on blood pressure. In general, Angelica gigas Nakai is a traditional herb that has been used for food and medicine for a long time because of its excellent hematopoietic, anticancer, antioxidant, and anti-inflammatory actions. However, direct use of the Angelica gigas extract has the problem of inducing high blood pressure according to the preparation thereof. In this Example, the Angelica extracts obtained in Examples 1-1 and 1-2 and 99.9% pure decursinol of the present disclosure were administered to rats and assayed for safety for blood pressure.

Using a blood pressure analyzer (3R229 3 channel rat system, Life Science), rats were measured for blood pressure while being acclimated to blood measurement operation. When the blood pressure analyzer is being operated, laboratory rats are startled by the sound of air pressure and rise in blood pressure. In order to eliminate this effect, after adaptation by repeatedly measuring 5 times a day, the experiment was performed when the blood pressure was kept stable, and the blood pressure was regularly measured every day even during the experiment to adapt the rats to the analyzer. When measuring blood pressure, the head was covered with a handkerchief to block light for stabilization. Fifteen experimental rats were divided into 5 groups of 3 rats for the next administration experiment and measured for blood pressure. A control (physiological saline), the Angelica extracts, and decursinol were each orally administered for two weeks, and the blood pressures measured the next day after the final administration are given in Table 1.

TABLE 1

| | Control | | Angelica Extract | | Decursinol (99%) | |
|---|---|---|---|---|---|---|
| | | Dias- | | Dias- | | Dias- |
| Dose | Systolic | tolic | Systolic | tolic | Systolic | tolic |
| 0.0 mg/kg | 121 | 115 | 120 | 115 | 125 | 118 |
| 0.5 mg/kg | 111 | 104 | 130 | 110 | 110 | 91 |
| 1.0 mg/kg | 110 | 102 | 135 | 105 | 110 | 92 |
| 2.0 mg/kg | 126 | 121 | 142 | 122 | 117 | 92 |
| 5.0 mg/kg | 123 | 115 | 140 | 120 | 115 | 90 |

As can be seen in Table 1, the control group ranged in systolic blood pressure from 110 mmHg to 126 mmHg. For the Angelica extract-administered group, the blood pressure was raised to at least 130 and up to 142 mmHg at a dose of 0.5 mg/Kg or higher. It was thus observed that the administration of Angelica gigas extract may cause the significant side effect of increasing blood pressure if not purified. When The 99 w/w % decursinol prepared in Example 1-2 was administered in the same manner, the systolic blood pressures of the rats were rather lowered to at least 100 mmHg and down to 117 mmHg, as shown in Table 1, indicating that the decursinol is free of the serious side effect exhibited upon the administration of Angelica gigas extract.

### Example 4: Inhibitory Effect of Decursinol on Vascular Smooth Muscle Cell Growth

### Example 4-1. High Sugar-Induced Excessive Growth of Vascular Smooth Muscle Cell

It was examined whether a high level of glucose increased the growth of the vascular smooth muscle cell line A10. A10 cells were seeded at a density of 1×10³ cells/ml into 96-well plates and incubated for 16 hours. Then, the medium was changed into a medium containing 5, 10, 20, or 30 mM D-glucose. After incubation for 72 hours, viable cells were counted by MTT assay. For 72 hours, A10 cells significantly proliferated with the increase of glucose concentration.

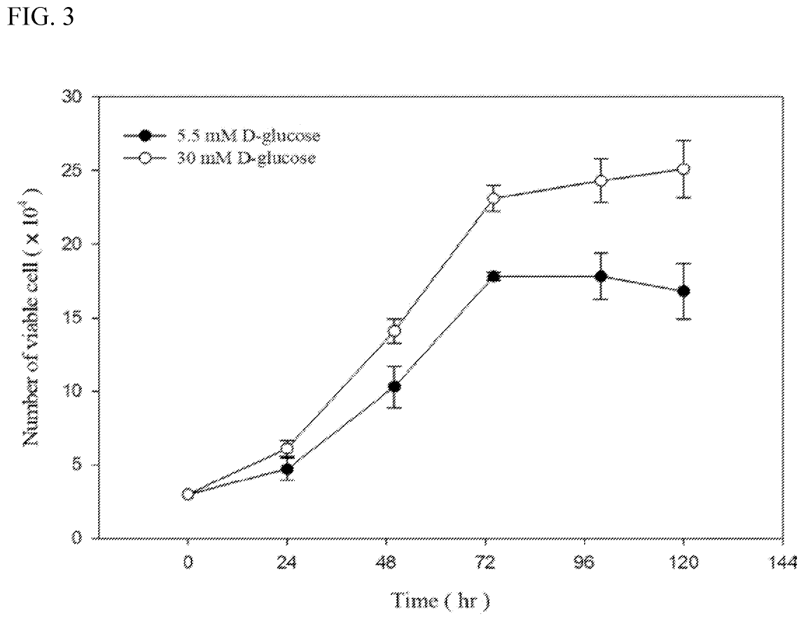

Based on the experiment data, the concentration of 30 mM D-glucose was determined as a hyperglycemia condition. Cell proliferation was compared with that at a normal D-glucose concentration. A10 cells were loaded at a density of 1×10³ cells/ml into 96-well plates, and after 16 hours of incubation, the medium was replaced with a medium containing a 5.5 or 30 mM D-glucose concentration. After 24, 72, 96, and 120 hours, viable cells were counted by MTT assay and the results are depicted in FIG. 3. As shown in FIG. 3, the proliferation of A10 cells was significantly increased in the medium of 30 mM D-glucose concentration. Cells cultured on high glucose showed a longer stationary phase than cells cultured on normal glucose, and as a result, cells in high glucose proliferated by about 20% to 25% more than cells in normal glucose.

### Example 4-2. Inhibition of Excessive Growth of Vascular Smooth Muscle Cells by Decursinol

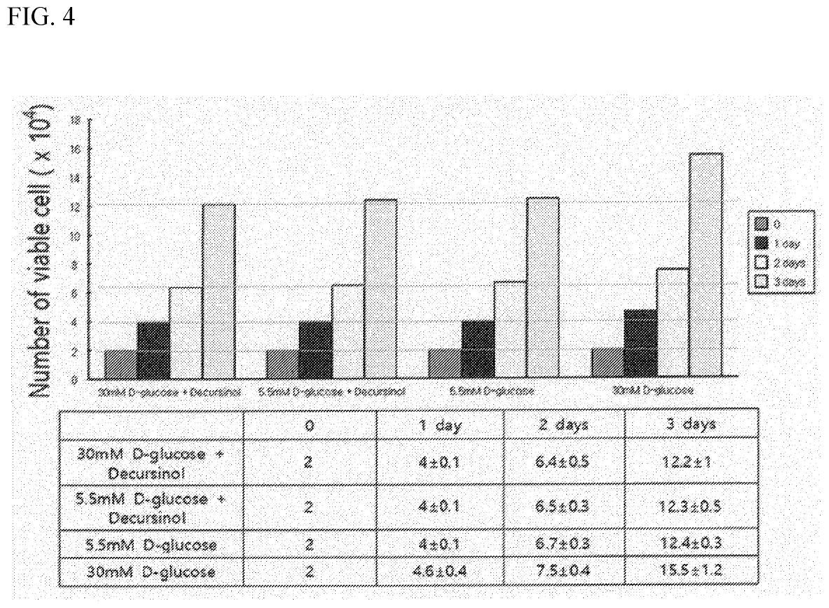

The effect of decursinol on the high sugar-induced proliferation of A10 cells was investigated. A10 cells were loaded at a density of 1×10³ cells/ml into 96-well plates, and after 16 hours, the medium was replaced with a medium containing a 5.5 or 30 mM D-glucose concentration. Then, each well was treated with 25 μM of solubilized decursinol, and the cells were counted after 1, 2, and 3 days. The results are depicted in FIG. 4.

As shown in FIG. 4, 25 μM decursinol did not significantly inhibit the proliferation of A10 cells at the normal sugar concentration of 5.5 mM D-glucose. However, the decursinol significantly inhibited the growth of A10 cells that had been excessively proliferated in a medium of 30 mM D-glucose, and on day 3, the proliferation of A10 cells was inhibited by about 20%.

In order to examine the cytotoxicity of decursinol, both live and dead cells were counted. After treatment with decursinol, 98% or more of live cells were observed alive. Consequently, it was found that when decursinol was applied to the cells in a normal glucose concentration medium, the proliferation of vascular smooth muscle cells was not affected, whereas when applied to high glucose, decursinol inhibited cell proliferation in a dose-dependent manner.

### Example 4-3. Inhibitory Effect of Decursinol on Phosphate-Protein Kinase

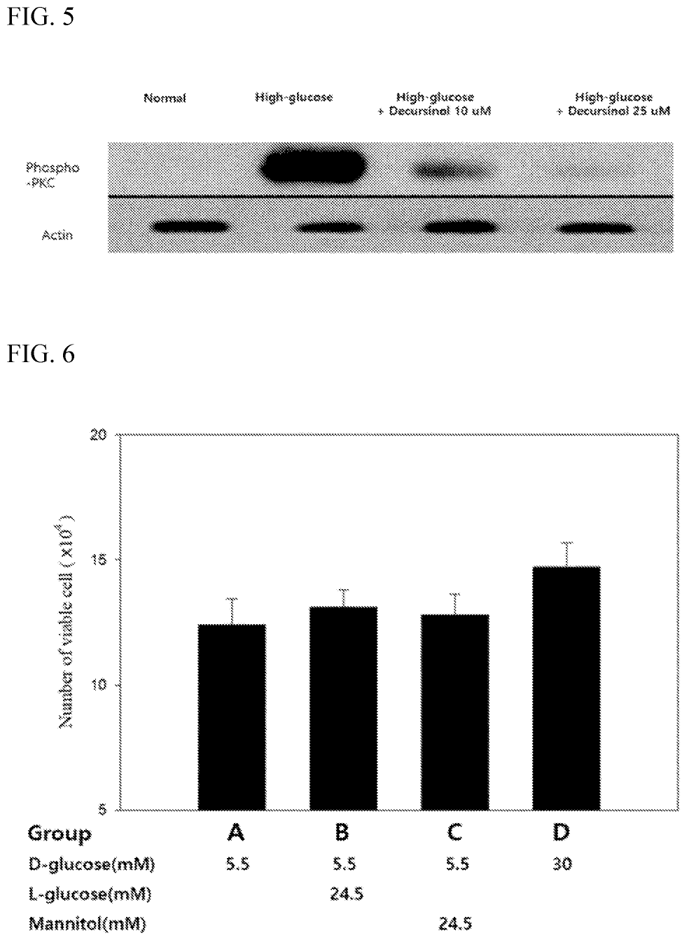

Examination was made to see whether the inhibitory effect of decursinol on high sugar-induced proliferation of A10 cells was associated with PKC. A10 cells were loaded at a density of 1×10³ cells/ml into 96-well plates, and after 16 hours, the medium was replaced with a medium containing a 5.5 or 30 mM D-glucose concentration. Then, each well was treated with 10 and 25 μM of solubilized decursinol and incubated for 24 hours. Western blotting was carried out using a pan-phospho-PKC antibody and the results are depicted in FIG. 5. As shown in FIG. 5, the expression level of phospho-PKC protein in A10 cells in a high sugar environment was significantly increased compared to that in a normal sugar environment. At this time, when applied for 24 hours, decursinol significantly decreased the increased expression level of phospho-PKC in high sugar in a dose-dependent manner.

From the above results, it was found that decursinol inhibited the high sugar-induced proliferation of A10 cells by reducing the expression level of phospho-PKC protein and thus lowering the PKC activity.

### Example 4-5. Observation of Effect of Osmotic Pressure on A10 Cell Growth

It was further examined whether the proliferation of A10 cells in a high-sugar environment was simply attributed to an osmotic pressure. A10 cells were seeded at a density of 3×10⁴ cells per well into 6-well plates and incubated overnight. Thereafter, the medium was exchanged with the sugar compositions shown in FIG. 6 and cultured for three days. The sugar compositions were designed to be four different groups. The cells were treated only with D-glucose (5.5 mM) for group A, with D-glucose (5.5 mM)+L-glucose (24.5 mM) for group B, with D-glucose (5.5 mM)+Mannitol (24.5 mM) for group C, and only with D-glucose (30 mM) for group D. Each experiment was conducted in triplicate. Viable cells in the culture cells were counted using the trypan blue exclusion method and the results are depicted in FIG. 6.

As shown in FIG. 6, group D, treated with 30 mM D-glucose, overgrew, but the groups added with mannitol or L-glucose, which is not metabolized in A10 cells, did not show a significant difference in cell number compared to the control group. Therefore, it was observed that A10 cell growth by the high sugar of 30 mM D-glucose was not affected by osmotic pressure.

### Example 4-6. Therapeutic Index of Decursinol, Decursin, and Decursinol Angelate

Decursinol, decursin, and decursinol angelate account for 10% of Angelica roots, making up the majority of the solid content. Among them, 54% is accounted for by decursin, 44% by decursinol angelate, which is an isomer of decursin, and 2% by decursinol. However, based on the fact, found in Example 3, that the Angelica extract exhibits certain side effects depending on the purification method therefor and that the side effects such as induction of hypertension do not appear when administering decursinol with a purity of 99 w/w % or higher, decursin, decursinol angelate, and decursinol were examined for cytotoxicity and growth inhibitory effect on A10 cells in high glucose so as to find which one, among decursin, decursinol angelate, and decursinol, has little cytotoxicity in a normal glucose level and suppresses the high glucose-induced proliferation of A10 cells and to detect its concentration.

The vascular smooth cell line A10 was loaded at a density of 1×10³ cells/ml into 96-well plates and after 16 hours, the medium was replaced with a medium having a concentration of 5.5 mM or 30 mM D-glucose. Subsequently, decursin, decursinol angelate and decursinol were each applied at concentrations of 0, 20, 50, 100, 200, and 500 μM to the cells. IC50, a concentration at which the growth of the cells is inhibited by 50%, and EC100, a concentration which inhibits the growth of cells in a high sugar level environment to a normal cell population, were measured, and therapeutic indices were calculated by dividing IC50 by EC100. The results are summarized in Table 2, below. As shown in Table 2, decursinol has an IC50 value of 500 μM or more, exhibiting almost no cytotoxicity. Then, effects of decursin and decursinol angelate to inhibit the proliferation of A10 cells in high glucose were expressed as EC100. EC100 refers to the concentration at which high glucose-induced proliferation of A10 cells is inhibited to normal glucose levels by the drug. The EC100 of decursinol angelate was 4.4 μM, with the highest growth inhibitory effect. That is, the growth inhibitory effect appeared in the order of decursinol angelate>decursin>decursinol. However, with respect to the therapeutic index calculated in consideration of toxicity, the effective substances for inhibiting the high glucose-induced proliferation of A10 cells at low concentrations, with almost no cytotoxicity under normal conditions were in the order of decursinol>decursinol angelate>decursin. The therapeutic index was calculated to be 11.5 for decursin, 28.1 for decursinol angelate, and 34.7 for decursinol, indicating that decursinol was the best.

In other words, decursinol angelate, although having a good inhibitory effect on smooth muscle proliferation in a high-sugar environment, exhibits cytotoxicity. It can be seen that decursin, co-existing as an isomer of decursinol angelate, is more likely to exhibit side effects due to cytotoxicity than decursinol angelate. This is consistent with the fact that, as demonstrated in Example 3, the Angelica extract containing larger contents of decursin and decursinol angelate exhibited side effects of inducing hypertension.

Therefore, it was found that both effects and safety were obtained by converting decursin and decursinol angelate contained in the Angelica extract to high-purity decursinol through the process as in Example 1-2.

TABLE 2

| | IC50 (μM) | EC100 (μM) | Therapeutic index* |
|---|---|---|---|
| Decursin | 82.7 | 7.2 | 11.5 |
| Decursinol | 123.5 | 4.4 | 28.1 |
| angelate | | | |
| Decursinol | >500 | 10.1 | >49.5 |

*Therapeutic index was defined as IC50/EC100

### Formulation Example 1: Pharmaceutical Formulation

### Formulation Example 1-1. Preparation of Tablet

200 mg of the decursinol-solubilized mixture of the present disclosure was mixed with 175.9 g of lactose, 180 g of potato starch, and 32 g of colloidal silicate. This mixture was added with a 10% gelatin solution and crushed before passage through a 14-mesh sieve. The fine mixture thus obtained was dried, added with 160 g of potato starch, 50 g of talc, and 5 g of magnesium, and prepared into tablets.

### Formulation Example 1-2. Preparation of Injection

100 mg of the decursinol-solubilized mixture of the present disclosure, 0.6 g of sodium chloride, and 0.1 g of ascorbic acid were dissolved in distilled water to make a volume of 100 ml. This solution was loaded into a vial and sterilized by heating at 20° C. for 30 minutes.

### Formulation Example 2: Preparation of Food

### Formulation Example 2-1. Preparation of Cooking Seasoning

A cooking seasoning for health promotion was prepared by adding the decursinol solubilized mixture of the present disclosure in an amount of 1% by weight to a cooking seasoning.

### Formulation Example 2-2. Preparation of Flour Food

The decursinol-solubilized mixture of the present disclosure was added in an amount of 0.1% by weight to wheat flour, and the resulting flour mixture was prepared into breads, cakes, cookies, crackers and noodles for health promotion.

### Formulation Example 2-3. Preparation of Soup and Gravies

The decursinol-solubilized mixture of the present disclosure was added in an amount of 0.1% by weight to broth to prepare a soup and gravies for health promotion.

### Formulation Example 2-4. Preparation of Dairy Products

The decursinol-solubilized mixture of the present disclosure was added in an amount of 0.1% by weight to milk which was then used to prepare various dairy products including butter and ice cream.

### Formulation Example 2-5. Preparation of Vegetable Juice

The decursinol-solubilized mixture of the present disclosure was added to 1,000 ml of tomato juice or carrot juice to prepare a vegetable juice for health promotion.

### Formulation Example 2-6. Preparation of Fruit Juice

The decursinol-solubilized mixture of the present disclosure was added in an amount of 0.1 g to 1,000 ml of apple juice or grape juice to prepare a fruit juice for health promotion.

1. A composition comprising decursinol as an active ingredient for prevention or treatment of a smooth muscle cell proliferative disease.

2. The composition of claim 1, wherein the composition is a pharmaceutical composition prepared through a process comprising: a first step of mixing decursinol with a detergent to give a first mixture; a second step of adding ethanol to the first mixture and mixing chitosan therewith to give a second mixture; a third step of dissolving the second mixture by adding a basic amino acid and purified water thereto, followed by adjusting the pH of the solution into 8.6 to give a third mixture; and a fourth step of sterilizing the third mixture.

3. The composition of claim 2, wherein the detergent in the first step is at least one selected from the group consisting of sodium dodecyl sulfate, polyoxyethylene sorbitan monooleate (Tween-80), polyoxyethylene sorbitan monostearate (Tween-60), polyoxyethylene sorbitan monopalmitate (Tween-40), polyoxyethylene sorbitan monolaurate (Tween-20), triton X-100, and nonyl phenoxypolyethoxylethanol (NP-40).

4. The composition of claim 2, wherein the water-soluble chitosan in the second step has a molecular weight of 20,000 or less.

5. The composition of claim 2, wherein the basis amino acid in the third step is arginine, lysine, or a mixture thereof.

6. The composition of claim 1, wherein the smooth muscle cell proliferative disease is at least one selected from the group consisting of angina pectoris, arteriosclerosis, cerebral infarction, benign prostatic hyperplasia, and hypertension.

7. A health functional food comprising decursinol as an active ingredient for prevention or alleviation of a smooth muscle cell proliferative disease.

8. The health functional food of claim 7, wherein the composition for inhibiting proliferation of smooth muscle cells is a pharmaceutical composition prepared through a process comprising: a first step of mixing decursinol with a detergent to give a first mixture; a second step of adding ethanol to the first mixture and mixing chitosan therewith to give a second mixture; a third step of dissolving the second mixture by adding a basic amino acid and purified water thereto, followed by adjusting the pH of the solution into 8.6 to give a third mixture; and a fourth step of sterilizing the third mixture.

9. The health functional food of claim 8, wherein the detergent in the first step is at least one selected from the group consisting of sodium dodecyl sulfate, polyoxyethylene sorbitan monooleate (Tween-80), polyoxyethylene sorbitan monostearate (Tween-60), polyoxyethylene sorbitan monopalmitate (Tween-40), polyoxyethylene sorbitan monolaurate (Tween-20), triton X-100, and nonyl phenoxypolyethoxylethanol (NP-40).

10. The health functional food of claim 8, wherein the water-soluble chitosan in the second step has a molecular weight of 20,000 or less.

11. The health functional food of claim 8, wherein the basis amino acid in the third step is arginine, lysine, or a mixture thereof.

12. The health functional food of claim 7, wherein the smooth muscle cell proliferative disease is at least one selected from the group consisting of angina pectoris, arteriosclerosis, cerebral infarction, benign prostatic hyperplasia, and hypertension.

13. A method for prevention or treatment of a smooth muscle cell proliferative disease, comprising administering a pharmaceutically effective amount of the composition of claim 1 to a subject in need thereof.

14. The method of claim 13, wherein the composition is a pharmaceutical composition prepared through a process comprising: a first step of mixing decursinol with a detergent to give a first mixture; a second step of adding ethanol to the first mixture and mixing chitosan therewith to give a second mixture; a third step of dissolving the second mixture by adding a basic amino acid and purified water thereto, followed by adjusting the pH of the solution into 8.6 to give a third mixture; and a fourth step of sterilizing the third mixture.

15. The method of claim 14, wherein the detergent in the first step is at least one selected from the group consisting of sodium dodecyl sulfate, polyoxyethylene sorbitan monooleate (Tween-80), polyoxyethylene sorbitan monostearate (Tween-60), polyoxyethylene sorbitan monopalmitate (Tween-40), polyoxyethylene sorbitan monolaurate (Tween-20), triton X-100, and nonyl phenoxypolyethoxylethanol (NP-40).

16. The method of claim 14, wherein the water-soluble chitosan in the second step has a molecular weight of 20,000 or less.

17. The method of claim 14, wherein the basis amino acid in the third step is arginine, lysine, or a mixture thereof.

18. The method of claim 13, wherein the smooth muscle cell proliferative disease is at least one selected from the group consisting of angina pectoris, arteriosclerosis, cerebral infarction, benign prostatic hyperplasia, and hypertension.

19. A process for preparing a composition comprising decursinol as an active ingredient, comprising a first step of mixing decursinol with a detergent to give a first mixture; a second step of adding ethanol to the first mixture and mixing chitosan therewith to give a second mixture; a third step of dissolving the second mixture by adding a basic amino acid and purified water thereto, followed by adjusting the pH of the solution into 8.6 to give a third mixture; and a fourth step of sterilizing the third mixture.

20. The process of claim 19, wherein the detergent in the first step is at least one selected from the group consisting of sodium dodecyl sulfate, polyoxyethylene sorbitan monooleate (Tween-80), polyoxyethylene sorbitan monostearate (Tween-60), polyoxyethylene sorbitan monopalmitate (Tween-40), polyoxyethylene sorbitan monolaurate (Tween-20), triton X-100, and nonyl phenoxypolyethoxylethanol (NP-40).
